(19) United States
(12) Patent Application Publication
(10) Pub. No.: US 2016/0296536 A1
(43) Pub. Date: Oct. 13, 2016

(54) Administration of Glucocorticoid Steroids to Livestock

(71) Applicant: Animal Biotech, LLC, Dallas, TX (US)

(72) Inventor: John J. McGLONE, Lubbock, TX (US)

(21) Appl. No.: 15/096,888

(22) Filed: Apr. 12, 2016

Related U.S. Application Data
(60) Provisional application No. 62/146,815, filed on Apr. 13, 2015.

(51) Int. Cl. A61K 31/573; A61K 9/00

(52) U.S. Cl. CPC A61K 31/573; A61K 9/0019

(57) ABSTRACT

There is provided a method of treating a weaning piglet with a cortisol agonist where the method includes administering a dose of the cortisol agonist to the weaning piglet, in a therapeutically effective amount, to cause an increase in body mass as compared to a like weaning but untreated piglet. The cortisol agonist may be dexamethasone. Further, the dose may range from about 0.1 mg/kg body mass to about 1.0 mg/kg of body mass, administered via intra-muscular injection. In a particular exemplary embodiment, the method includes administering a dose of 0.2 mg/kg body mass via intra-muscular injection. The method may include administering a first dose before weaning, and administering a second dose after weaning. Further, the first dose may be administered a day before commencing weaning, and the second dose 3 days after weaning has commenced.

### CROSS REFERENCE TO RELATED APPLICATION

This application claims the benefit of provisional application U.S. Ser. No. 62/146,815, filed on Apr. 13, 2015, entitled “Administration of Glucocorticoid Steroids to Increase Livestock Weight Gain.”

### BACKGROUND

1. Field of the Invention

The invention relates to the field of livestock rearing, and more particularly to administration of a glucocorticoid steroid agonist to enhance the growth rate (mass gain) of the livestock animal at the stressful post-weaning stage and other times of stress, to increase the mass gain per unit of feed, and also to improve blood antioxidant level, blood interleukin-1β (iβ-1β) level, and blood haptoglobin levels, which indicate health benefits.

2. Description of the Related Art

In the livestock industry, an important economic driver is the weight gain of an animal over time, and the weight gain for a given amount of feed eaten. A higher weight gain in farm animals is economically positive. In addition, if the feed required to produce a unit (pound or kilogram) of weight gain is reduced, this has positive economic and environmental consequences.

There are unavoidable stressful times in the life of a livestock animal when weight gain might be compromised. For example, it has been found that the weaning stage of a piglet is particularly significant in that it interrupts a desired rate of growth (in weight) of the piglet. It has been speculated that weaning in itself imposes stress on the piglet, and this stress in turn results in a decreased rate of weight gain. The same could be said for other stresses like social stress, handling and transportation.

In modern pig farming, which is a large-scale controlled enterprise where costs and benefits are continually assessed, an interval of reduced weight gain during the weaning process is significant in adding to the costs of producing pork. During this weaning interval, despite that the piglets are fed and that their health is managed, weight gain is subpar overall and reduced per unit of feed intake. Because of this, the overall time period until they are within a size or weight range to be harvested, is extended. This also reduces the capacity of the farm by reducing the turnaround time of the farm. Capacity increases then require an extension of buildings and related equipment, which adds capital costs. If the turnaround time (i.e. the time for the piglets to reach harvest size) is reduced, more piglet-raising cycles can be obtained within the same farm, without the need for additional capital investment. Thus, the weaning interval of lowered weight gain has cost implications as to direct costs (such as feed), indirect costs (such as labor and utilities) as well as capital required.

There is a significant financial incentive to improve weight gain and weight gain per unit of feed during the weaning period to effectively reduce time to achieve the harvest weight. But thus far efforts have not yielded results that may be regarded as uniformly successful and that provide a positive cost-benefit analysis.

### SUMMARY

The following is a summary of some aspects and exemplary embodiments of the present technology, of which a more detailed explanation is provided under the Detailed Description section, here below.

An exemplary embodiment provides a method of treating a weaning piglet with a cortisol agonist where the method includes administering a dose of the cortisol agonist to the weaning piglet, in a therapeutically effective amount, to cause an increase in body mass as compared to a like weaning but untreated piglet. The cortisol agonist may be dexamethasone. Further, the dose may range from about 0.1 mg/kg body mass to about 1.0 mg/kg of body mass, administered via intra-muscular injection. In a particular exemplary embodiment, the method includes administering a dose of 0.2 mg/kg body mass via intra-muscular injection. The method may include administering a first dose before weaning, and administering a second dose after weaning. Further, the first dose may be administered a day before commencing weaning stress, and the second dose 3 days after weaning has commenced.

An exemplary embodiment provides a method of treating a weaned or stressed pig with a cortisol agonist where the method includes administering the cortisol agonist to the weaning piglet, in a therapeutically effective amount, to cause an increase in body mass gain and body weight per unit of feed compared to a like but untreated piglet. The cortisol agonist may be dexamethasone. Further, the dose may range from about 0.1 mg/kg body mass to about 1.0 mg/kg of body mass, administered via intra-muscular injection. In a particular exemplary embodiment, the method includes administering a dose of 0.2 mg/kg body mass via intra-muscular injection. The method may include administering a first dose before weaning, and administering a second dose after weaning. Further, the first dose may be administered a day before commencing weaning, and the second dose 3 days after weaning has commenced.

An exemplary embodiment provides a method of treating a weaning piglet with a cortisol agonist, where the method includes administering the cortisol agonist to the weaning piglet, in a therapeutically effective amount, to cause a reduction in blood haptoglobin level as compared to the weaning but untreated piglet. The cortisol agonist may be dexamethasone. Further, the dose may range from about 0.1 mg/kg body mass to about 1.0 mg/kg of body mass, administered via intra-muscular injection. In a particular exemplary embodiment, the method includes administering a dose of 0.2 mg/kg body mass via intra-muscular injection. The method may include administering a first dose before weaning, and administering a second dose after weaning. Further, the first dose may be administered a day before commencing weaning stress, and the second dose 3 days after weaning has commenced.

An exemplary embodiment provides a method of treating a weaning piglet with a cortisol agonist, where the method includes administering the cortisol agonist to the weaning piglet, in a therapeutically effective amount, to cause a reduction in blood interleukin-1β (iβ-1β) level as compared to the weaning but untreated piglet. The cortisol agonist may be dexamethasone. Further, the dose may range from about 0.1 mg/kg body mass to about 1.0 mg/kg of body mass, administered via intra-muscular injection. In a particular exemplary embodiment, the method includes administering a dose of 0.2 mg/kg body mass via intra-muscular injection. The method may include administering a first dose before weaning, and administering a second dose after weaning. Further, the first dose may be administered a day before commencing weaning, and the second dose 3 days after weaning has commenced.

The foregoing is a brief summary of some aspects of the technology presented herein, and does not limit the scope of the inventions, which are described in the claims here below

### BRIEF DESCRIPTION OF THE DRAWINGS

FIG. 1 is a bar chart representation of body weight (BW) for each group of piglets in the study.

FIG. 2 is a series of graphs depicting the body weight (BW) of piglets over time with treatment by drug administration, compared to a control.

FIG. 3 shows the effect of administration of drugs on body weight (BW) of piglets at 35 days, compared to control piglets.

FIG. 4 illustrates by a bar graph the serum cortisol level in piglet blood samples before and 24 hours after weaning.

FIG. 5 illustrates a bar graph of antioxidant level in unweaned piglets, weaned piglets, and weaning piglets treated with serum cortisol.

FIG. 6 illustrates a bar graph of interleukin-1β (iL-1β) level in unweaned piglets, weaned piglets, and weaning piglets treated with a cortisol agonist.

FIG. 7 illustrates a bar graph of average body mass gain for unweaned piglets, weaned piglets, and weaning piglets treated with a cortisol agonist, on one Y-axis, and average daily mass gain to feed ratio on the other Y-axis.

FIG. 8 is a series of graphs showing haptoglobin level on the y-axis versus days after weaning for unweaned piglets, weaned piglets, and weaning piglets treated with a cortisol agonist.

FIG. 9 is a Table of data corresponding to Example 1.

### DETAILED DESCRIPTION

The following provides a detailed description of exemplary embodiments of the particle-free microdermabrasion formulations. It should be understood that describing examples of these embodiments facilitates an understanding of the inventions, but the exemplary embodiments do not limit the scope of the inventions in any way. The inventions are demarcated only by the claims appended here below.

With regard to terminology, the terms “weight gain” and “mass gain” may be used interchangeably herein and have the same meaning, namely, a gain in mass of an animal.

It is well-known that the weaning stage of a piglet is particularly significant in that it interrupts a desired rate of growth (in weight) of the piglet. It has been speculated that weaning in itself imposes stress on the piglet, and this stress in turn results in a decreased rate of weight gain or even a weight loss. These stresses may relate for example to a loss of the maternal scent (pheromones) or a loss maternal-neonatal or social behavior patterns established prior to weaning. Regardless of the source(s) and nature of the stresses during weaning, the piglet loses mass, or daily mass gain is reduced, and/or mass gain per unit of feed intake is reduced.

There have been attempts in the past to treat livestock animals with glucocorticoid steroids. These were for various purposes, but none were directed specifically at weaning piglets to address the reduced body mass gain (and even loss of mass) from the actual weaning at 2 to 5 weeks of age and during the post-weaning period of up to about 4 weeks of age and until market at about 6 months of age. Indeed, there has been no suggestion that a cortisol agonist, including but not limited to cortisol, corticosterone, dexamethasone or any glucocorticoid agonist (a molecule that has glucocorticoid effects for example, would counter the deleterious effect of weaning on the body mass gain of piglets. It is in fact surprising that giving a stress hormone such as a glucocorticoid agonist might reduce the negative effects of a stress. To treat animals at a time of stress with a stress hormone is counter intuitive.

It was hypothesized that, contrary to expectations, administering a therapeutically effective dosage of a cortisol agonist would have a beneficial effect on the mass gain and mass gain per unit of feed in weaning piglets. Stress is believed to reduce weight gain and have other negative effects on growth and health in part due to a rise in stress hormones like the glucocorticoids. That administering a stress hormone, in this case any cortisol agonist, at a time of stress would benefit an animal's growth or health is unexpected but highly positive and beneficial to animals.

Tests were conducted on the effect of treating weaning piglets with a cortisol agonist. Contrary to expectations that this might enhance stress in the weaning piglets, and have a further detrimental effect on their mass, it was found that the piglets experienced either a reduction in the extent of expected mass loss, or experienced a gain in mass. These results were surprising in view of the ordinary expectations, as explained above.

In exemplary embodiments, the mass gain of weaning piglets treated with a cortisol agonist in accordance with the inventive technology is from about 4 to 15% over a period of 4 to 20 weeks, as compared to like weaning piglets that were not so treated. In some exemplary embodiments, the mass gains were in the range from about 3 to about 10% over a period of 28 days after weaning, compared to like weaning piglets that were not so treated. These mass gains are significant in terms of the economics of livestock industrial farming. Additionally, increasing weight gain and body weight 4 weeks after weaning is expected to result in a heavier pig at market time (presently about 5 to 7 months of age).

Moreover, in exemplary embodiments, the mass gain per unit of feed intake of weaning piglets treated with a cortisol agonist in accordance with the inventive technology is from about 4 to about 15% per kg of feed over a period of at least 28 days compared to like weaning piglets that were not so treated. In some exemplary embodiments, the mass gains were in the range from about 3 to about 10% per kg of feed over a period of at least 28 days, as compared to like weaning piglets that were not so treated. These mass gains per unit of feed intake are significant in terms of the economics of livestock industrial farming.

The following examples illustrate aspects of the technology but do not limit the inventions described herein that are encompassed in the patent claims.

### EXAMPLE 1

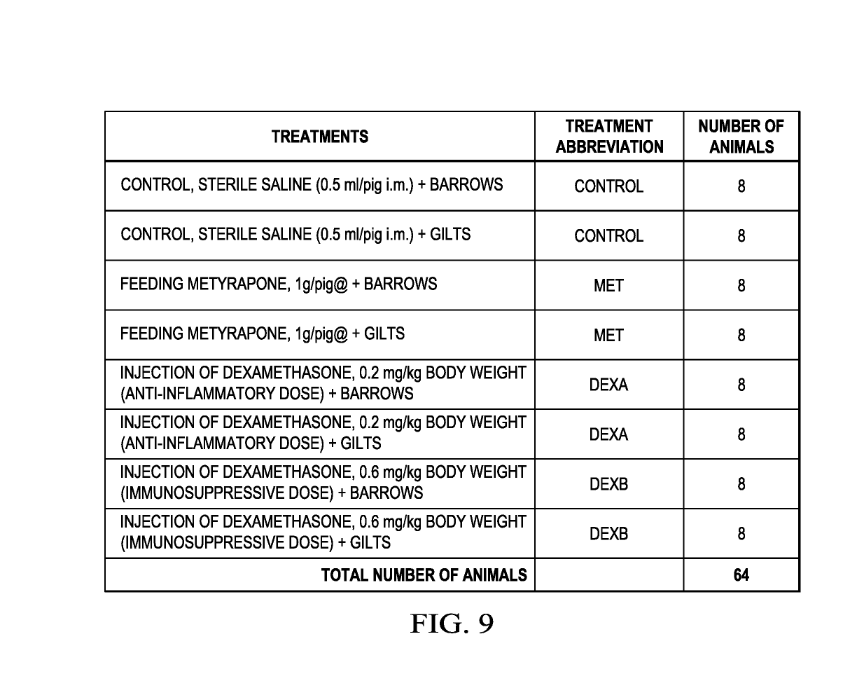

In a study, a total of 64 piglets (32 gilts [females] and 32 barrows [castrated males]) with initial body weight (BW) of 4.8±0.7 Kg were assigned to four treatments. Treatments were in a 4×2 factorial arrangement with a completely randomized design with two main factors: (I) control (Con), two levels of cortisol agonist (Dexamethasone; DEX; subdivided into 2 dosage levels: DEXA and DEXB), or cortisol antagonist (Metyrapone; MET), and (II) gender (gilt vs. barrow). See Table at FIG. 9, for a summary of the list of the treatments, drug dosage, and number of the animals per treatment. Dexamethasone was injected intramuscular at dosages shown, while Metyrapone was mixed with applesauce and fed to the animals with a syringe.

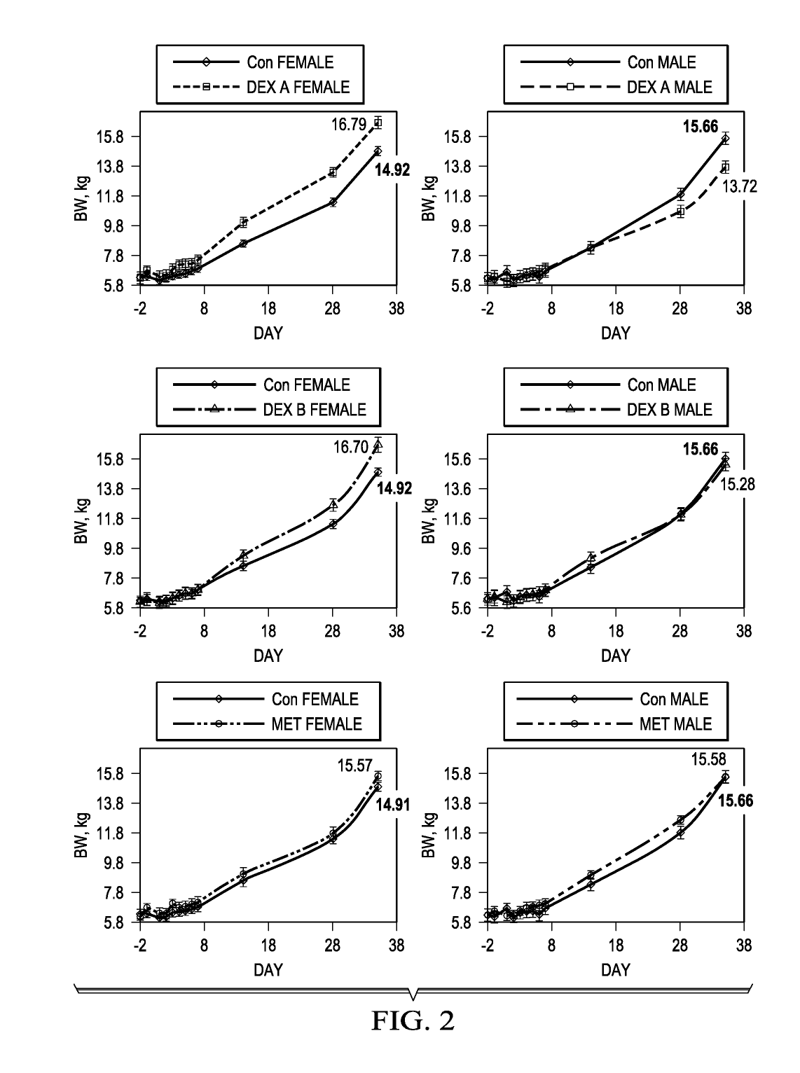

The study lasted for 5 weeks. It began one week before weaning (14-day old piglets), and continued 4 weeks after the weaning. Pigs were weaned at the age of 21-days. Piglets received the treatments either orally via diet or by intra-muscular injection one week before and again 24 hours before weaning. Blood samples were collected by venipuncture of the jugular vein on day 14, 20, 21, 22, 23, 24, 26, 28, and 35 of age. Serum cortisol was determined using a commercial kit. Feed intake, average daily gain, feed efficiency, and mortality/morbidity was determined. Body weights (body mass) of the animals were measured on a daily basis for a week after weaning, and then on a weekly basis until the end of the study. See, FIGS. 2 and 3.

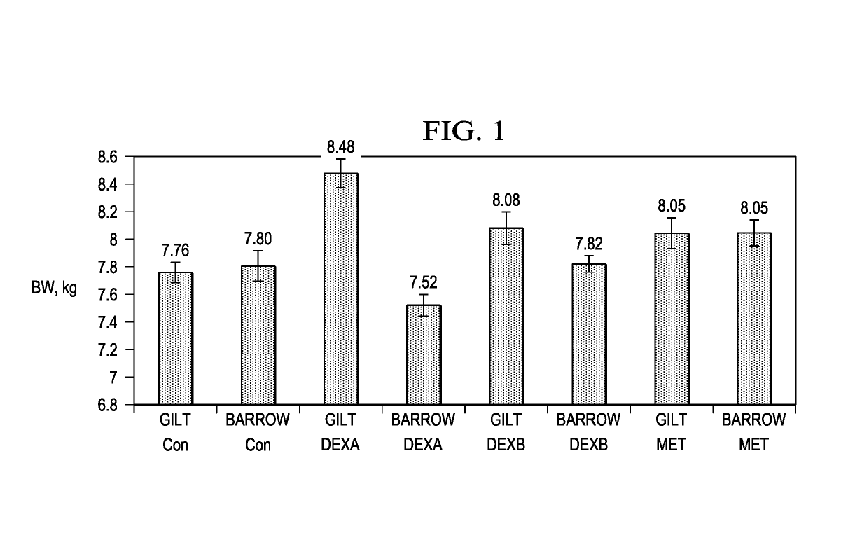

It was found that relative to control animals, feeding MET resulted in higher body weights in both gilts and barrows (4 and 3%, respectively; FIG. 1) P<0.01). MET is a cortisol antagonist. Studies have reported a substantial increase in cortisol in blood of weaning piglets, believed to be caused by stress during weaning. Weaning is potentially the most stressful time in a piglet's life. Thus, it is theorized without being bound that the improved body mass in MET-treated piglets can likely be associated with suppressive effects of MET on cortisol synthesis.

Dexamethasone (a corticosteroid) improved the body weight in female pigs at the both doses (9.3 and 4.1%, at DEXA and DEXB, respectively; FIG. 1; P<0.01). At anti-inflammatory dose, dexamethasone had no effect on body weight of barrows (P>0.05; FIG. 1). It has been reported in the literature that weaning induces intestinal inflammation in piglets. Therefore, improved body weights of gilts might be attributed to the anti-inflammatory/immunosuppressive effects of dexamethasone. At day 35 of the study only DEXA and DEXB gilts had a higher body weight than the controls (13 and 12%, respectively; FIGS. 2 and 3; P<0.05).

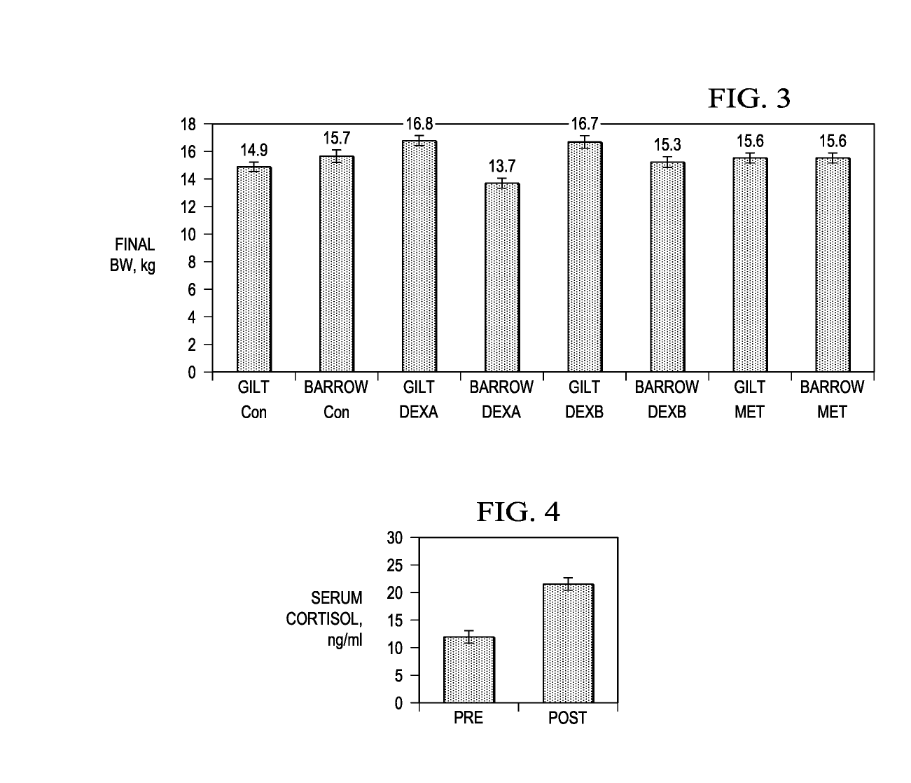

The “Feed to Weight Gain” ratio was not affected by the treatments (P<0.05). Weaning resulted in increased serum cortisol levels (P<0.0; FIG. 4). Neither MET nor DEX had an effect on serum cortisol levels at 24 h after weaning (P>0.05).

Both cortisol agonist (DEXA and B) and antagonist (MET) reduced the negative impact of weaning on average body weight of the piglets during the course of the study. However, the effect of dexamethasone on body weight of gilts was more pronounced and lasted longer compared to MET. MET increased the average body weight of the piglets by 3.4%, but had no effect on body weight of piglets at the day 35 of the study. In comparison, dexamethasone, at both anti-inflammatory and immunosuppressive doses, significantly improved the average body weight of the gilts (9.3 and 4.4%, respectively). Based on this study, dexamethasone is a better choice than metyrapone for reducing the negative impact of weaning on the mass gain performance of piglets.

One might have speculated that blocking the stress hormone cortisol's synthesis may improve animal growth and health; however, it was found in other tests that doing so had negative effects. That the opposite (adding glucocorticoids at a time of stress) has benefits, is surprising in view of what might have been expected.

Other tests were also conducted to determine the health and body mass performance of weaning piglets, as detailed in Example 2, here below.

### EXAMPLE 2

In this test, eighteen gilts (of commercially relevant genetics) were weaned at 23±2 days of age. These were housed in floor pens (3 pigs per pen), and assigned to 3 treatments:

(1) unweaned suckling that received saline but no cortisol agonist (UWS),

(2) weaning that received only saline (WS), and

(3) weaning that received the cortisol agonist (WC).

The piglets of Group (3) received a cortisol agonist (0.2 mg dexamethasone per kg of body mass) via intramuscular injection on the day before commencing weaning, and day 3 post-weaning. Those of Groups (1) and (2) received instead only a saline solution intra-muscular injection at the same time intervals.

Body mass (BW) was monitored daily, and average daily mass gain (ADG) and mass of feed intake (ADFI) were measured and/or estimated for the course of the study.

Blood samples were collected via jugular puncture on day 0, and on day 1, day 4, and day 5 post-weaning. Blood was assayed for interleukin-1β (IL-1β), antioxidant, and haptoglobin (Hp) levels.

At the end of the study the piglets were euthanized and a section of the jejunum was collected for histological examination. Data was analyzed using mixed procedures in SAS (PROC MIXED). Repeated measure analysis of variance was used for the parameters that were measured over time.

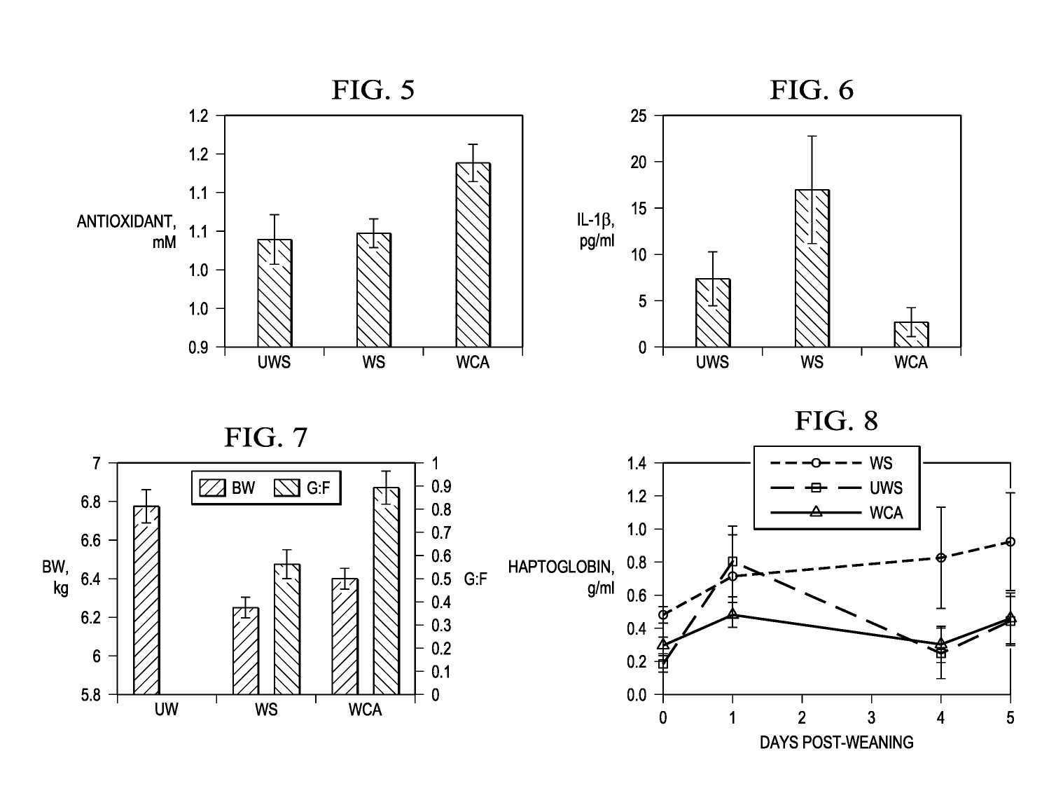

The results are illustrated graphically in FIGS. 5-8.

From the results it is apparent that body mass (BW) was higher in WCA relative to WS, but it was lower than UWS pigs (FIG. 7; P<0.04). Thus, there was a positive impact on mass gain for treated weaning piglets, but not so much as to fully compensate for weaning.

Also, from FIG. 7, in the WCA weaned pigs there was a higher mass gain-to-feed (G:F) ratio (FIG. 1; P<0.01).

In weaned pigs CA treatment had no effect on ADG and ADFI (Table 1; P<0.01).

WCA pigs had higher antioxidant levels than both WS and UWS (FIG. 1; P<0.02)

In weaned pigs WCA the cortisol agonist treatment reduced plasma Hp (FIG. 8) and IL-1β (FIG. 6) as compared to WS (FIG. 1; P<0.04).

It was also found that the cortisol agonist treatment did not improve intestinal villi architecture in jejunum compared to WS (P>0.42). The stress of segregated early weaning reduced the villus height by 28±2% in piglets; (P<0.01), but had no effect on crypt depth.

It was also found that antioxidant levels were higher in the treated (WCA) piglets than in the WS and UWS piglets, as seen in FIG. 5.

While at least one exemplary embodiment has been presented in the foregoing detailed description section, it should be appreciated that many variations exist. It should also be appreciated that the exemplary embodiments are only examples, and are not intended to limit the scope, applicability, or configuration of the claimed inventions in any way. Rather, the foregoing detailed description provides a convenient road map of an enabling description for those of ordinary skill in the art to implement the illustrative exemplary embodiments, among others. It should be understood that various changes can be made in the function and arrangement of elements described herein without departing from the scope of the patent claims listed below, including the legal equivalents of these patent claims.

1. A method of treating a weaning piglet with a cortisol agonist, the method comprising administering a dose of the cortisol agonist to the weaning piglet in a therapeutically effective amount to cause an increase in body mass compared to the weaning but untreated piglet.

2. The method of claim 1, wherein the cortisol agonist is dexamethasone.

3. The method of claim 2, wherein the administering comprising administering a dose of from 0.1 mg/kg body mass to 1.0 mg/kg of body mass via intra-muscular injection.

4. The method of claim 3, comprising administering a first dose before weaning and administering a second dose after weaning.

5. The method of claim 4, comprising administering the first dose a day before commencing weaning, and administering the second dose 3 days after weaning has commenced.

6. The method of claim 2, wherein the administering comprising administering a dose of 0.2 mg/kg body mass via intra-muscular injection.

7. A method of treating a weaning piglet with a cortisol agonist, the method comprising administering the cortisol agonist to the weaning piglet in a therapeutically effective amount to cause an increase in body mass gain per unit of feed compared to the weaning but untreated piglet.

8. The method of claim 7, wherein the cortisol agonist is dexamethasone.

9. The method of claim 8, wherein the administering comprising administering a dose of from 0.1 mg/kg body mass to 1.0 mg/kg of body mass via intra-muscular injection.

10. The method of claim 9, comprising administering a first dose before weaning and administering a second dose after weaning.

11. The method of claim 10, comprising administering the first dose a day before commencing weaning, and administering the second dose 3 days after weaning has commenced.

12. The method of claim 8, wherein the administering comprising administering a dose of 0.2 mg/kg body mass via intra-muscular injection.

13. A method of treating a weaning piglet with a cortisol agonist, the method comprising administering the cortisol agonist to the weaning piglet in a therapeutically effective amount to cause a reduction in blood haptoglobin level as compared to the weaning but untreated piglet

14. The method of claim 13, wherein the cortisol agonist is dexamethasone.

15. The method of claim 14, wherein the administering comprising administering a dose of from 0.1 mg/kg body mass to 1.0 mg/kg of body mass via intra-muscular injection.

16. The method of claim 15, comprising administering a first dose before weaning and administering a second dose after weaning.

17. The method of claim 16, comprising administering the first dose a day before commencing weaning, and administering the second dose 3 days after weaning has commenced.

18. The method of claim 14, wherein the administering comprising administering a dose of 0.2 mg/kg body mass via intra-muscular injection.

19. A method of treating a weaning piglet with a cortisol agonist, the method comprising administering the cortisol agonist to the weaning piglet in a therapeutically effective amount to cause a reduction in blood interleukin-1β (iL-1β) level as compared to the weaning but untreated piglet.

20. The method of claim 19, wherein the cortisol agonist is dexamethasone.

21. The method of claim 20, wherein the administering comprising administering a dose of from 0.1 mg/kg body mass to 0.5 mg/kg of body mass via intra-muscular injection.

22. The method of claim 21, comprising administering a first dose before weaning and administering a second dose after weaning.

23. The method of claim 22, comprising administering the first dose a day before commencing weaning, and administering the second dose 3 days after weaning has commenced.

24. The method of claim 20, wherein the administering comprising administering a dose of 0.2 mg/kg body mass via intra-muscular injection.

25. A method of treating a weaning piglet with a cortisol agonist, the method comprising administering the cortisol agonist to the weaning piglet in a therapeutically effective amount to cause an increase in blood antioxidant content as compared to the weaning but untreated piglet.

26. The method of claim 25, wherein the cortisol agonist is dexamethasone.

27. The method of claim 26, wherein the administering comprising administering a dose of from 0.1 mg/kg body mass to 1.0 mg/kg of body mass via intra-muscular injection.

28. The method of claim 27, comprising administering a first dose before weaning and administering a second dose after weaning.

29. The method of claim 28, comprising administering the first dose a day before commencing weaning, and administering the second dose 3 days after weaning has commenced.

30. The method of claim 26, wherein the administering comprising administering a dose of 0.2 mg/kg body mass via intra-muscular injection.
